(19) United States
(12) Patent Application Publication
(10) Pub. No.: US 2004/0141929 A1
(43) Pub. Date: Jul. 22, 2004

(54) Composition

(75) Inventor: Peter John Hall, Merseyside (GB)

Correspondence Address: UNILEVER, PATENT DEPARTMENT, 45 RIVER ROAD, EDGEWATER, NJ, 07020, US

(73) Assignee: Unilever Home & Personal Care USA, Division of Conopco, Inc.

(21) Appl. No.: 10/719,137

(22) Filed: Nov. 21, 2003

(30) Foreign Application Priority Data
Nov. 22, 2002 (EP) 02258069.0
Feb. 21, 2003 (EP) 03251047.1

(51) Int. Cl. A61K 7/16

(52) U.S. Cl. 424/49

(57) ABSTRACT

An oral composition comprising a compound of Formula 1:

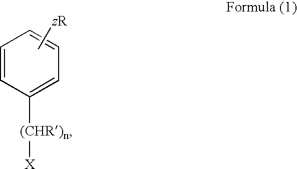

wherein:

at least one R is a metal or ammonium salt of —OH, the remaining R groups independently selected from the group consisting of: H, F, Cl, Br, —OH, C1 to C₅-alkyl, —C(O)H, and —C(O)—C1 to C₅-alkyl and z takes a value of from 1 to 5;

R′ is selected from the group consisting of: H, —OH, F, Cl, Br, I, and C₁-C₆ alkyl and n is an integer of from 0 to 12;

wherein X is a group selected from —C(O)—NH—R″, —R″, —C(O—R″, —C(O)O—R″, and —SO₂—R″; and R″ is selected from the group consisting of: —C₅₋₁₆ alkyl or —CH₂C₆H₆.

### BACKGROUND OF THE INVENTION

1. Field of the Invention

The present invention relates to an oral composition comprising a salt of an alkyl hydroxybenzoate.

2. Related Art

Salts of alkyl hydroxybenzoates (parabens) are known in the art where the alkyl group is methyl, ethyl, propyl or butyl in The Handbook of Pharmaceutical Excipients, A. H Kibbe ed, Pharmaceutical Press, London.

We have found that there exists a range of compounds which exhibit surprisingly high antibacterial efficacy and are not disclosed for use in oral compositions in the prior art.

### BRIEF DESCRIPTION OF THE INVENTION

Accordingly, the invention provides an oral composition comprising a compound of Formula 1:

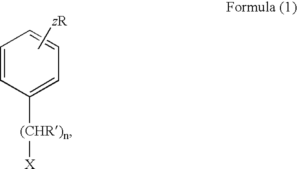

wherein:

at least one R is a metal or ammonium salt of —OH, the remaining R groups independently selected from the group consisting of: H, F, Cl, Br, —OH, C₁ to C₅-alkyl, —C(O)H, and —C(O)—C₁ to C₅-alkyl and z is from 1 to 5;

R′ is selected from the group consisting of: H, —OH, F, Cl, Br, I, and C₁-C₆ alkyl and n is an integer of from 0 to 12;

wherein X is a group selected from —C(O)—NH—R″, —R″, —C(O—R″, —C(O)O—R″, and —SO₂—R″ and R″ is selected from the group consisting of: —C₅₋₁₂ alkyl or —CH₂C₆H₆.

### DETAILED DESCRIPTION OF THE INVENTION

In a preferred embodiment X is —C(O)O—R″, wherein R″ is a substituted or unsubstituted branched or straight chain hydrocarbon moiety comprising from 5 to 16 and especially from 7 to 10 carbon atoms. Examples of suitable R″ groups include pentyl, hexyl, benzyl, heptyl, octyl, 2-ethyl hexyl, nonyl, decyl, undecyl, dodecyl and tridecyl. Of these the most preferred are the straight chain alkyls. The most preferred active is where R″ is n-octyl.

The key feature of the active according to Formula 1 is that it comprises a metal salt of one of the OH groups represented by R. Where this metal has a valency of more than 1 as many of the molecules of Formula 1 as is required to function as a counter ion will exist.

Preferably the metal is an alkali metal selected from Group Ia of the Periodic Table or an alkaline earth metal selected from Group IIa. However, the salt counterion may also be an ammonium group.

Wherein the metal is an alkali metal at least one R will be selected from the group consisting of: —OK, —ONa, and, —OLi, preferably —ONa.

According to Formula 1 z is from 1 to 5 and is preferably 1 or 2, more preferably 1.

According to Formula 1 R′ is selected from the group consisting of: H, —OH, F, Cl, Br, I, and C₁-C₆ alkyl.

Manufacture of such compounds as represented by Formula 1 would be a simple step for the man skilled in the art to carry out.

The most preferred antimicrobial agent is the sodium salt of n-octyl parahydroxy benzoate because it has the greatest antimicrobial effect against the commonly present oral microflora.

Further preferred embodiments include the following aspects of compounds represented by Formulas 2 to 5:

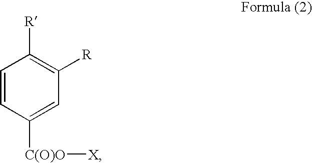

wherein R is either OH or Cl and R′ is either H or OH, wherein X is either a substituted or unsubstituted, straight chain or branched alkyl group having from 2 to 16 carbon atoms. At least one of the R or R′ groups is a salt of an OH group as described above.

In a preferred embodiment where, in Formula (2), R is Cl it is preferred that R′ is a salt of an OH group.

Preferably the alkyl group in Formula (2) is an aliphatic alkyl group, more preferably comprising from 1 to 16 and especially from 3 to 12 carbon atoms. Examples of suitable alkyl groups include methyl, ethyl, propyl, isopropyl, butyl, pentyl, hexyl, benzyl, heptyl, octyl, 2-ethyl hexyl, nonyl, decyl, undecyl, dodecyl or tridecyl. Of these the most preferred are the straight chain alkyls. The most preferred compound is where the alkyl group is n-octyl.

In the most preferred case the compound of Formula (2) is a metal or ammonium salt of 3-hydroxybenzoic acid octyl ester; 3-chloro, 4-hydroxybenzoic acid octyl ester or 3, 4-dihydroxybenzoic acid octyl ester. Most preferably, it is a metal or ammonium salt of one of or a mixture of 3-hydroxybenzoic acid octyl ester and 3-chloro, 4-hydroxybenzoic acid octyl ester.

A second preferred aspect to the invention provides an oral care composition comprising a compound of Formula (3)

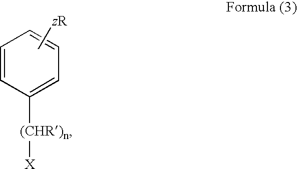

wherein:

R is a group independently selected from the group consisting of: H, F, Cl, Br, —OH, C₁₋₅ alkyl, —C(O)H, —C(O)C₁₋₅ alkyl, —OCH₃, —C₂H₅, —NH₂, —NHC(O)CH₃ and C(O)OC₁₋₆alkyl and z is from 1 to 5;

R′ is selected from the group consisting of: H, —OH, F, Cl, Br, I, and C₁-C₆ alkyl and n is an integer of from 0 to 12;

at least one of R or R′ being a metal or ammonium salt of a OH group;

wherein X is —C(O)—R″ and R″ is —C₁₋₁₆ alkyl or —CH₂C₆H₆.

In a preferred embodiment R″ is a substituted or unsubstituted branched or straight chain hydrocarbon moiety comprising from 1 to 16 and especially from 5 to 10 carbon atoms. Examples of suitable R″ groups include pentyl, hexyl, benzyl, heptyl, octyl, 2-ethyl hexyl, nonyl, decyl, undecyl, dodecyl and tridecyl. Of these the most preferred are the straight chain alkyls. The most preferred active is where R″ is n-octyl.

According to Formula (3) z is from 1 to 5 and can be any number in between. Preferably z is 1.

According to Formula (3) R′ is selected from the group consisting of: H, —OH, F, Cl, Br, I, and C₁-C₆ alkyl. Preferably R′ is OH.

According to Formula (3) n is an integer of from 0 to 12. Preferably n is zero.

Preferably, at least one R group is in the para position.

Preferably, R is OH (salt as described herein or free OH), more preferably, in the para position.

In the most preferred case the compound of Formula (3) is a metal or ammonium salt of 1-(4-hydroxyphenyl)nonan-1-one.

According to a third preferred aspect the invention provides an oral care composition comprising a compound of Formula (4)

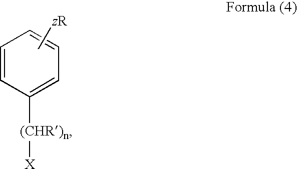

wherein:

R is a group independently selected from the group consisting of: H, F, Cl, Br, —OH, C₁₋₅ alkyl, —C(O)H, —C(O)C₁₋₅ alkyl, —OCH₃, —C₂H₅, —NH₂, —NHC(O)CH₃ and C(O)OC₁₋₆ alkyl and z is from 1 to 5;

R′ is selected from the group consisting of: H, —OH, F, Cl, Br, I, and C₁-C₆ alkyl and n is an integer of from 0 to 12;

at least one of R or R′ being a metal or ammonium salt of a OH group;

wherein X is —SO₂NH—R″ and R″ is —C₁₋₁₆ alkyl or —CH₂C₆H₆.

In a preferred embodiment R″ is a substituted or unsubstituted branched or straight chain hydrocarbon moiety comprising from 1 to 16 and especially from 5 to 10 carbon atoms. Examples of suitable R″ groups include pentyl, hexyl, benzyl, heptyl, octyl, 2-ethyl hexyl, nonyl, decyl, undecyl, dodecyl and tridecyl. Of these the most preferred are the straight chain alkyls. The most preferred active is where R″ is n-octyl.

According to Formula (4) z is from 1 to 5, preferably 3. Preferably R is Cl or OH. More preferably and where z is 3, there are two R groups as Cl and one R group as OH. In this embodiment it is preferred that the two Cl groups are in positions 3 and 5 while the OH group is in position 6.

Preferably, n is zero.

Most preferably, the compound of Formula (4) is a metal or ammonium salt of 3,5-dichloro, 2-hydroxy, N-octylbenzene sulphonamide.

According to a fourth preferred aspect the invention provides an oral care composition comprising a compound of Formula (5)

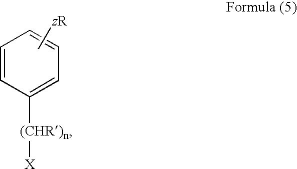

wherein:

R is a group independently selected from the group consisting of: —OH, C(O)OC₁₋₁₆ alkyl and z is from 1 to 5;

R′ is selected from the group consisting of: H, —OH, F, Cl, Br, I, and C₁-C₆ alkyl and n is an integer of from 0 to 12;

at least one of R or R′ being a metal or ammonium salt of a OH group;

wherein X is —C(O)O—R″ and R″ is —C₁₋₁₆ alkyl or —CH₂C₆H₆.

In a preferred embodiment R or R″ is, independently from one another, a substituted or unsubstituted branched or straight chain hydrocarbon moiety comprising from 1 to 16 and especially from 1 to 8, more preferably 3 to 4 carbon atoms. Of these the most preferred are the straight chain alkyls. The most preferred active is where R″ is n-butyl. Preferably R and R″ are the same.

According to Formula (5) z is from 1 to 5 and can be any number in between. Preferably z is 2.

According to Formula (5) R′ is selected from the group consisting of: H, —OH, F, Cl, Br, I, and C₁-C₆ alkyl and n is an integer of from 0 to 12. Preferably n is zero.

The most preferred compound of Formula (5) is a metal or ammonium salt of 5-hydroxy isophthalic acid dibutyl ester.

In a second aspect the invention provides the use of a compound according to any of Formulas 1 to 5 in an oral care composition as an antimicrobial agent. Such use may be as an anti-tartar agent, anti-caries agent, anti-oral malodour agent, anti-gingivitis agent and any other related use for an antimicrobial agent in an oral composition.

In a third aspect the invention provides the use of a compound according to any of Formulas 1 to 5 in the manufacture of a medicament for the treatment or prevention of any one or more of gingivitis, oral malodour, tartar, tooth plaque build-up and caries.

In a fourth aspect the invention provides the use of a compound according to any of Formulas 1 to 5 as described herein as a delivery enhancing agent in an oral care composition for a halogenated diphenyl ether, preferably triclosan.

The compound according to one of Formulas 1 to 5 is preferably present in an amount such that an antibacterial effect can be provided. In practice this ranges from 0.15 to 30% by weight of the composition according to the invention. Preferably, in an amount ranging from 0.2 to 10% by weight and even more preferably from 0.1 to 3.5% by weight.

The composition according to the invention may also comprise a divalent metal salt. Preferably, the divalent metal salt is a salt selected from the group consisting of zinc- and stannous salts such as zinc citrate, zinc sulphate, zinc glycinate, sodium zinc citrate, stannous pyrophosphate and mixtures thereof. The preferable divalent metal salt is zinc citrate.

Suitably, the amount of divalent metal salt ranges from 0.01 to 10% by weight of the composition, preferably from 0.05 to 5% by weight, more preferably from 0.1 to 2% by weight and especially preferably from 0.3 to 0.9% by weight of the composition.

The oral composition according to the invention comprise further ingredients which are common in the art, such as:

antimicrobial agents, e.g. Triclosan, chlorhexidine, sanguinarine extract, metronidazole, quaternary ammonium compounds, such as cetylpyridinium chloride; bis-guanides, such as chlorhexidine digluconate, hexetidine, octenidine, alexidine; and halogenated bisphenolic compounds, such as 2,2′ methylenebis-(4-chloro-6-bromophenol);

anti-inflammatory agents such as ibuprofen, flurbiprofen, aspirin, indomethacin etc.;

anti-caries agents such as sodium- and stannous fluoride, aminefluorides, sodium monofluorophosphate, sodium trimeta phosphate and casein;

plaque buffers such as urea, calcium lactate, calcium glycerophosphate and strontium polyacrylates;

vitamins such as Vitamins A, C and E;

plant extracts;

desensitising agents, e.g. potassium citrate, potassium chloride, potassium tartrate, potassium bicarbonate, potassium oxalate, potassium nitrate and strontium salts;

anti-calculus agents, e.g. alkali-metal pyrophosphates, hypophosphite-containing polymers, organic phosphonates and phosphocitrates etc.;

biomolecules, e.g. bacteriocins, antibodies, enzymes, etc.;

flavours, e.g. peppermint and spearmint oils;

proteinaceous materials such as collagen;

preservatives;

opacifying agents;

colouring agents;

pH-adjusting agents;

sweetening agents;

pharmaceutically acceptable carriers, e.g. starch, sucrose, water or water/alcohol systems etc.;

surfactants, such as anionic, nonionic, cationic and zwitterionic or amphoteric surfactants;

particulate abrasive materials such as silicas, aluminas, calcium carbonates, dicalciumphosphates, calcium pyrophosphates, hydroxyapatites, trimetaphosphates, insoluble hexametaphosphates and so on, including agglomerated particulate abrasive materials, usually in amounts between 3 and 60% by weight of the oral care composition.

humectants such as glycerol, sorbitol, propyleneglycol, xylitol, lactitol etc.;

binders and thickeners such as sodium carboxymethylcellulose, xanthan gum, gum arabic etc. as well as synthetic polymers such as polyacrylates and carboxyvinyl polymers such as Carbopol®;

polymeric compounds which can enhance the delivery of active ingredients such as antimicrobial agents can also be included;

buffers and salts to buffer the pH and ionic strength of the oral care composition; and

other optional ingredients that may be included are e.g. bleaching agents such as peroxy compounds e.g. potassium peroxydiphosphate, effervescing systems such as sodium bicarbonate/citric acid systems, colour change systems, and so on.

Liposomes may also be used to improve delivery or stability of active ingredients.

The oral compositions may be in any form common in the art, e.g. toothpaste, gel, mousse, aerosol, gum, lozenge, powder, cream, etc. and may also be formulated into systems for use in dual-compartment type dispensers.

The present invention also provides a method of treating an oral condition selected from gingivitis, tartar, stained teeth, plaque, halitosis and mixtures thereof by brushing the teeth with a composition comprising a compound of Formula 1:

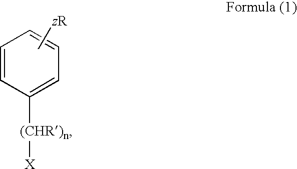

wherein:

at least one R is a metal or ammonium salt of —OH, the remaining R groups independently selected from the group consisting of: H, F, Cl, Br, —OH, C₁ to C₅-alkyl, —C(O)H, and —C(O)—C₁ to C₅-alkyl and z takes a value of from 1 to 5;

R′ is selected from the group consisting of: H, —OH, F, Cl, Br, I, and C₁-C₆ alkyl and n is an integer of from 0 to 12; wherein X is a group selected from —C(O)—NH—R″, —R″, —C(O—R″, —C(O)O—R″, and —SO₂—R″; and R″ is selected from the group consisting of: —C₅₋₁₆ alkyl or —CH₂C₆H₆.

The description and examples illustrate selected embodiments of the present invention. In light thereof variations and modifications will be suggested to one skilled in the art, all of which are within the spirit and purview of this invention.

Embodiments according to the invention shall now be discussed with reference to the following non-limiting examples.

### EXAMPLE

The following is a formulation according to the present invention. It is made by known processes.

| Ingredient | % w/w |
|---|---|
| 70% aq. sorbitol | 45.0 |
| Saccharin | 0.2 |
| Polyethylene glycol | 2.0 |
| Titanium dioxide | 1.0 |
| Sodium fluoride | 0.32 |
| Thickening silica | 9.0 |
| Abrasive silica | 10.0 |
| SLS | 1.6 |
| Sodium carboxymethylcellulose | 0.8 |
| Flavour | 1.0 |
| Zinc citrate trihydrate | 0.75 |
| Sodium salt n-Octyl paraben | 1.0 |
| Water | to 100 |

1. An oral composition comprising a compound of Formula 1: 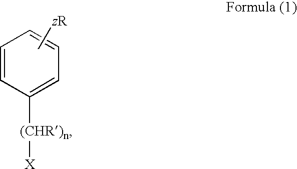
wherein:
at least one R is a metal or ammonium salt of —OH, the remaining R groups independently selected from the group consisting of: H, F, Cl, Br, —OH, C₁ to C₅-alkyl, —C(O)H, and —C(O)—C₁ to C₅-alkyl and z takes a value of from 1 to 5;
R′ is selected from the group consisting of: H, —OH, F, Cl, Br, I, and C₁-C₆ alkyl and n is an integer of from 0 to 12;
wherein X is a group selected from —C(O)—NH—R″, —R″, C(O—R″, —C(O)O—R″, and —SO₂—R″; and R″ is selected from the group consisting of: —C₅₋₁₆ alkyl or —CH₂C₆H₆.

2. An oral composition according to claim 1, wherein X is —C(O)O—R″.

3. An oral composition according to claim 1, wherein R″ is an aliphatic alkyl group.

4. An oral composition according to claim 1, wherein R″ represents a straight chain alkyl group comprising from 5 to 12 carbon atoms.

5. An oral composition according to claim 1, wherein the metal is selected from Group Ia of the Periodic Table.

6. An oral composition according to claim 5, wherein at least one R is selected from the group consisting of: —OK, —ONa, and, —OLi.

7. An oral composition according to claim 6, wherein at least one R is —ONa.

8. An oral composition according to claims 1, wherein the metal is selected from Group IIa of the Periodic Table.

9. An oral composition according to claim 1, wherein —R″ is C₆-C₁₂-alkyl.

10. An oral composition according claim 9, wherein —R″ is C₈-alkyl.

11. An oral composition according to claim 1, wherein z is 1.

12. An oral composition according to claim 1, wherein the compound of formula I is present in the composition in the range of from 0.001 to 5% by weight.

13. Oral composition according to claim 1 wherein the composition comprises an agent selected from the group consisting of anti-caries agents, anti-tartar agents, anti-oral malodour agents, tooth whitening agents, breath freshening agents and mixtures thereof.

14. A method of treating an oral condition selected from gingivitis, tartar, stained teeth, plaque, halitosis and mixtures thereof by brushing the teeth with a composition comprising a compound of Formula 1: 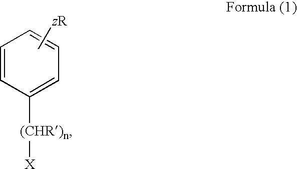
wherein:
at least one R is a metal or ammonium salt of —OH, the remaining R groups independently selected from the group consisting of: H, F, Cl, Br, —OH, C₁ to C₅-alkyl, —C(O)H, and —C(O)—C₁ to C₅-alkyl and z takes a value of from 1 to 5;
R′ is selected from the group consisting of: H, —OH, F, Cl, Br, I, and C₁-C₆ alkyl and n is an integer of from 0 to 12; wherein X is a group selected from —C(O)—NH—R″, —R″, —C(O—R″, —C(O)O—R″, and —SO₂—R″; and R″ is selected from the group consisting of: —C₅₋₁₆ alkyl or —CH₂C₆H₆.
